(19) United States
(12) Patent Application Publication
(10) Pub. No.: US 2013/0293847 A1
(43) Pub. Date: Nov. 7, 2013

(54) OPHTHALMOLOGIC APPARATUS

(71) Applicant: CANON KABUSHIKI KAISHA, Tokyo (JP)

(72) Inventor: Ryuji Tanaami, Yokohama-shi (JP)

(73) Assignee: CANON KABUSHIKI KAISHA, Tokyo (JP)

(21) Appl. No.: 13/873,830

(22) Filed: Apr. 30, 2013

(30) Foreign Application Priority Data
May 2, 2012 (JP) 2012-105364
Mar. 27, 2013 (JP) 2013-066883

(51) Int. Cl. A61B 3/00

(52) U.S. Cl. CPC A61B 3/0075
USPC 351/245; 351/246

(57) ABSTRACT

An ophthalmologic apparatus includes an ophthalmologic examination unit configured to examine a subject's eye, a movable unit at which the ophthalmologic examination unit is movably disposed, a driving force generation unit configured to generate a driving force to drive the movable unit, and a driving force transmission unit configured to reduce the driving force transmitted to the movable unit in a case where an external force is applied to the movable unit.

### BACKGROUND OF THE INVENTION

1. Field of the Invention

The present invention relates to an ophthalmologic apparatus configured to adjust a position thereof relative to a subject's eye, and, for example, examine the eye.

2. Description of the Related Art

As an ophthalmologic apparatus, there is a well-known apparatus including an approach prevention unit configured to detect a distance between a subject's eye and the apparatus, and prevent the apparatus from approaching the subject's eye beyond a predetermined distance. More specifically, Japanese Patent Application Laid-Open No. 8-126608 discusses a configuration that detects a position of an ophthalmologic examination unit relative to a fixed table when making a relative positional adjustment between the ophthalmologic examination unit and the subject's eye by moving a movable table with the ophthalmologic examination unit placed thereon in a front-back direction. Then, a movement limitation unit is actuated based on the detection result to prevent the ophthalmologic examination unit from abnormally approaching a subject's eye. In a case where the ophthalmologic apparatus is a non-contact tonometer, the ophthalmologic examination unit approaches the subject's eye until the ophthalmologic examination unit is approximately 10 mm away from the subject's eye. In this case, a range where the above-described abnormal approach can be detected is a range of approximately 10 mm from the subject's eye.

On the other hand, when the movable table moves closer to the subject, a subject's limb such as a hand may be caught between the fixed table and the movable table. At this time, it is necessary to prevent the movable table from further moving closer to the subject with the subject's limb kept caught between the fixed table and the movable table.

### SUMMARY OF THE INVENTION

According to an aspect of the present invention, an ophthalmologic apparatus includes an ophthalmologic examination unit configured to examine a subject's eye, a movable unit at which the ophthalmologic examination unit is movably disposed, a driving force generation unit configured to generate a driving force to drive the movable unit, and a driving force transmission unit configured to reduce the driving force transmitted to the movable unit in a case where an external force is applied to the movable unit.

In the ophthalmologic apparatus according to the present invention, it is possible to reduce the driving force transmitted to the movable unit based on the external force applied to the movable unit in a case where a subject's limb is caught between the movable unit and another portion of the ophthalmologic apparatus. As a result, it is possible to prevent the movable unit from further moving closer to the subject with the subject's limb kept caught between the movable unit and another portion.

Further features and aspects of the present invention will become apparent from the following detailed description of exemplary embodiments with reference to the attached drawings.

### BRIEF DESCRIPTION OF THE DRAWINGS

The accompanying drawings, which are incorporated in and constitute a part of the specification, illustrate exemplary embodiments, features, and aspects of the invention and, together with the description, serve to explain the principles of the invention.

FIG. 1 is a side view schematically illustrating a configuration of an ophthalmologic apparatus according to an exemplary embodiment of the present invention.

FIG. 2 is a perspective view schematically illustrating a configuration of a driving mechanism disposed at the ophthalmologic apparatus according to the exemplary embodiment of the present invention.

FIGS. 3A, 3B, and 3C are perspective views illustrating a configuration and an operation of a planetary gear mechanism disposed at the driving mechanism.

FIG. 4 is a perspective view schematically illustrating how a driving unit transmits a driving force via a first drive train.

FIG. 5 is a perspective view schematically illustrating how the driving unit transmits the driving force via a second drive train.

FIG. 6 is a perspective view schematically illustrating how the driving unit transmits the driving force via the second drive train with a sun gear fixed so as not to rotate.

FIG. 7 is a block diagram illustrating a configuration of a switching unit of the ophthalmologic apparatus according to the exemplary embodiment of the present invention.

FIG. 8 is a flowchart illustrating an operation of the ophthalmologic apparatus according to the exemplary embodiment of the present invention.

### DESCRIPTION OF THE EMBODIMENTS

Various exemplary embodiments, features, and aspects of the invention will be described in detail below with reference to the drawings.

### (Configuration of Ophthalmologic Apparatus)

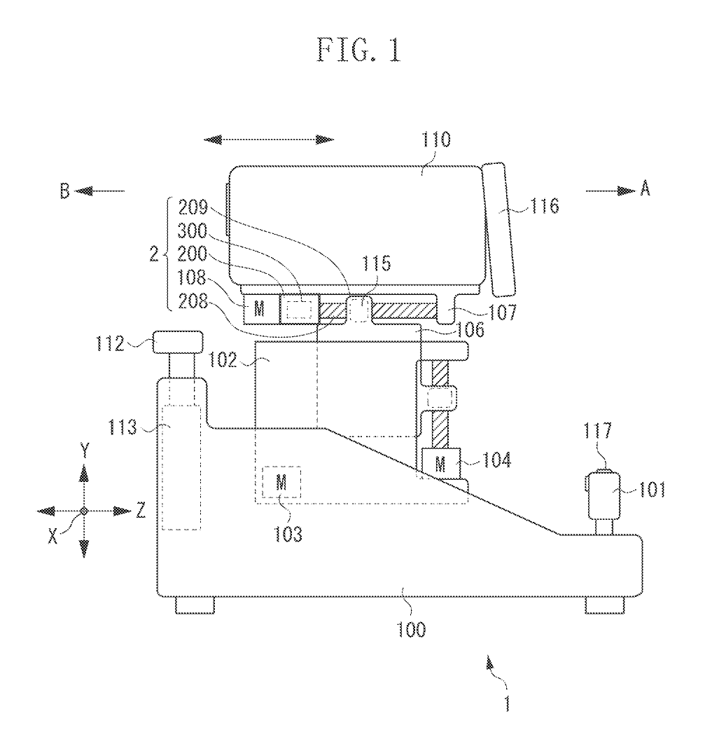

First, an entire configuration of an ophthalmologic apparatus 1 according to an exemplary embodiment of the present invention will be described with reference to FIG. 1. FIG. 1 is a side view schematically illustrating the configuration of the ophthalmologic apparatus 1 according to the present exemplary embodiment. An arrow A in FIG. 1 indicates a position where an examiner (an operator), who examines an eye, is situated. An arrow B in FIG. 1 indicates a position where a subject, whose eye is examined, is situated.

The ophthalmologic apparatus 1 includes a fixed table (a base) 100, an X movable table 102, a Y movable table 106, a Z movable table 107 as a movable unit, and a measurement unit 110 as an ophthalmologic examination unit.

The X movable table 102 is configured to be movable in a left-right direction (this direction is referred to as an X-axis direction) as viewed from the subject, whose eye is examined, relative to the fixed table 100. A motor 103 as a driving force generation unit, and a driving unit (not illustrated) configured to drive the X movable table 102 with use of the motor 103 are disposed between the fixed table 100 and the X movable table 102. The X movable table 102 moves in the X-axis direction by a driving force generated by the motor 103.

The Y movable table 106 is configured to be movable in a vertical direction (this direction is referred to as a Y-axis direction) as viewed from the subject, whose eye is examined, relative to the fixed table 100. A motor 104 as the driving force generation unit, and a driving unit (not illustrated) configured to drive the Y movable table 106 with use of the motor 104 are disposed between the Y movable table 106 and the X movable table 102. The Y movable table 106 moves in the Y-axis direction by a driving force of the motor 104.

The Z movable table 107 as the movable unit is configured to be movable in a front-back direction (this direction is referred to as a Z-axis direction) as viewed from the subject, whose eye is examined, relative to the fixed table 100. A driving mechanism 2, which includes a motor 108 as the driving force generation unit and a driving unit 200 configured to drive the Z movable table 107 with use of a driving force of the motor 108, is disposed between the Z movable table 107 and the Y movable table 106. The driving unit 200 converts the driving force generated by the motor 108 into a driving force for causing the Z movable table 107 to move in the Z-axis direction. The Z movable table 107 moves in the Z-axis direction by the driving force of the motor 108.

The driving mechanism 2 is configured in the following manner. The driving mechanism 2 includes the motor 108 as the driving force generation unit, the driving unit 200, a conveyance screw 208, and a nut 209. The motor 108 and the driving unit 200 are disposed at the Z movable table 107. The motor 108 as the driving force generation unit generates a driving force (rotational power). The driving unit 200 transmits the driving force generated by the motor 108 to the conveyance screw 208. The driving unit 200 includes a planetary gear mechanism 300 (the details thereof will be described below). The driving unit 200 can transmit the driving force generated by the motor 108 to the conveyance screw 208 via the planetary gear mechanism 300 and can also transmit the driving force generated by the motor 108 to the conveyance screw 208 without an intervention of the planetary gear mechanism 300.

The conveyance screw 208 is rotatably supported by the Z movable table 107 in such an orientation that an axis of the conveyance screw 208 extends in parallel with the Z-axis direction. The conveyance screw 208 rotates in response to the driving force transmitted from the motor 108 via the driving unit 200. The nut 209 is engaged with the conveyance screw 208, and is coupled to the Y movable table 106. The nut 209 moves on the conveyance screw 208 in the Z-axis direction according to a rotation of the conveyance screw 208. Therefore, when the conveyance screw 208 rotates, the Z movable table 107 moves in the Z-axis direction together with the nut 209.

The measurement unit 110 as the ophthalmologic examination unit is disposed at the Z movable table 107. When the Z movable table 107 moves, the measurement unit 110 moves in the Z-axis direction (i.e., in a direction toward and a direction away from a subject's eye to be examined) together with the Z movable table 107. The measurement unit 110 can examine the subject's eye by projecting a light flux onto the subject's eye. For example, the measurement unit 110 can measure an intraocular pressure of the subject's eye without contacting the subject's eye. However, an item that the measurement unit 110 can measure is not limited to the intraocular pressure of the subject's eye.

A monitor 116 as a notification unit is disposed at the measurement unit 110. The monitor 116 can be embodied by a display device such as a liquid crystal display device provided with a touch panel. The monitor 116 can display a captured image of the subject's eye, a menu for allowing the examiner to operate and set the measurement unit 110, and other predetermined information. The predetermined information displayed by the monitor 116 includes information for notifying the examiner that an external force is applied to the measurement unit 110 and the Z movable table 107 to prevent their movements (as will be described below).

A position detector 115 as a position detection unit is disposed between the Z movable table 107 and the Y movable table 106. The position detector 115 can detect a position of the measurement unit 110 and the Z movable table 107 in the Z-axis direction (a distance between the Z movable table 107 and the fixed table 100 and a chin support table 112 in the Z-axis direction, or a distance between the measurement unit 110 and the subject's eye) throughout an entire range where the Z movable table 107 can move in the Z-axis direction. The position detector 115 as the position detection unit can be embodied by, for example, a displacement meter capable of detecting a displacement amount based on a change in a resistance value or a displacement meter capable of detecting a displacement amount with use of laser.

Further, the chin support table 112, a chin support table driving mechanism 113 for driving the chin support table 112, and a joystick 101 are disposed at the fixed table 100. The chin support table 112 is a table where a chin of the subject is rested during an eye examination. The chin support table 112 can move in the vertical direction (the Y-axis direction) relative to the fixed table 100 by a driving force of the chin support table driving mechanism 113. The joystick 101 is an operation member used to operate the X movable table 102, the Y movable table 106, and the Z movable table 107. A button 117 is disposed at the joystick 101.

### <Configuration of Driving Unit>

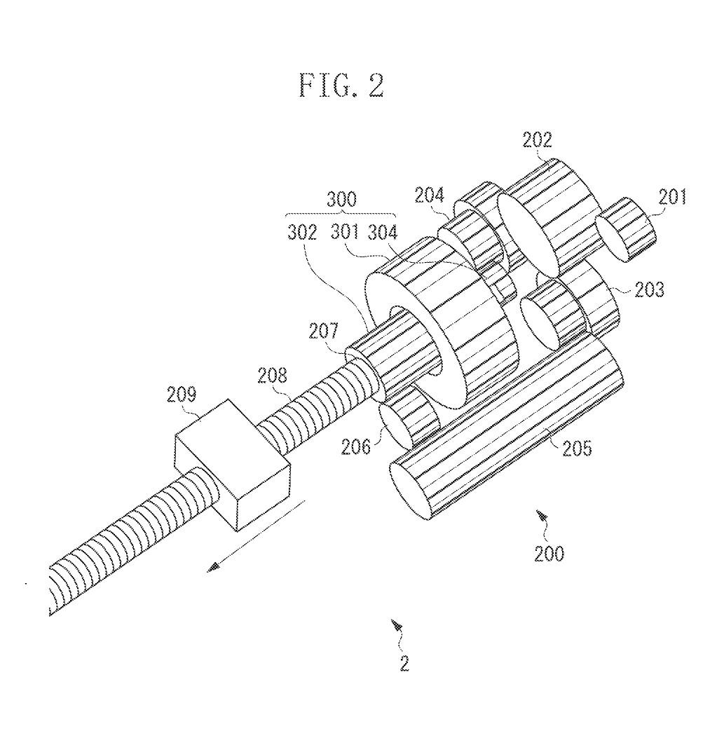

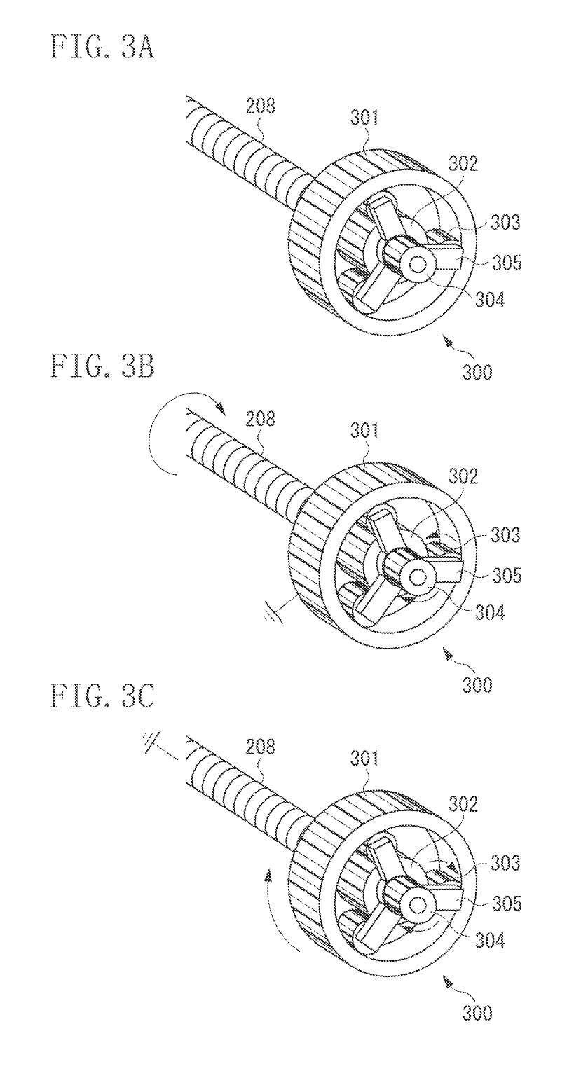

Next, a configuration of the driving unit 200 of the driving mechanism 2 disposed at the ophthalmologic apparatus 1 according to the present exemplary embodiment will be described with reference to FIGS. 2 and 3. FIG. 2 is a perspective view schematically illustrating the configuration of the driving unit 200. Further, FIG. 3A is a perspective view schematically illustrating a configuration of the planetary gear mechanism 300 included in the driving unit 200. Further, FIG. 3B is a perspective view schematically illustrating how power is transmitted to a sun gear by the planetary gear mechanism 300. Further, FIG. 3C is a perspective view schematically illustrating how power is transmitted to an internally-toothed gear by the planetary gear mechanism 300 in a state where driving of the conveyance screw 208 is blocked.

The driving unit 200 includes a pinion gear 201, a first gear 202, a first clutch 203 and a second clutch 204 as a switching unit, the planetary gear mechanism 300, a first idler gear 205, and a second idler gear 206. The planetary gear mechanism 300 is a kind of gear mechanism including a gear train. Examples of gear mechanisms include a speed reducer, a speed changer, and a differential gear mechanism, in addition to the planetary gear mechanism. Gear mechanisms are used for, for example, a speed reduction, a speed change, and distribution of power. The planetary gear mechanism 300 employed in the present exemplary embodiment includes a sun gear 302, a plurality of planetary gears 303, an internally-toothed gear 301, a carrier 305 (a planet carrier), and a carrier gear 304.

Generally, a planetary gear mechanism includes a sun gear, a plurality of planetary gears meshed with the sun gear, and an internally-toothed gear meshed with the plurality of planetary gears. The sun gear and the internally-toothed gear are coaxially disposed, and the planetary gears are disposed between the sun gear and the internally-toothed gear. The planetary gear mechanism is used as a speed reducer mechanism by, for example, inputting rotational power into any of the sun gear, the planetary gears, and the internally-toothed gear, and outputting rotational power from any of the other gears. Further, the planetary gear mechanism is used as a rotational power distribution mechanism by, for example, inputting rotational power into any of the sun gear, the planetary gears, and the internally-toothed gear, and outputting rotational power from the other two gears. In a case where the planetary gear mechanism is used as the rotational power distribution mechanism, rotational speeds of the two gears that output the rotational power automatically change according to loads applied to the respective gears. Even if one of the gears that output the rotational power is fixed, the other of the gears that output the rotational power and the gear to which the rotational power is input can continue their rotations.

In the planetary gear mechanism 300 employed in the exemplary embodiment of the present invention, externally-toothed gears are used as the sun gear 302 and the plurality of planetary gears 303. The sun gear 302 and the internally-toothed gear 301 are coaxially disposed, and the plurality of planetary gears 303 is disposed between the sun gear 302 and the internally-toothed gear 301. The plurality of planetary gears 303 is supported by the carrier 305 so as to be rotatable respectively and be revolvable about the sun gear 302. The sun gear 302 and the plurality of planetary gears 303 are meshed with each other, and the plurality of planetary gears 303 and the internally-toothed gear 301 are meshed with each other. Therefore, rotational power transmitted (input) to the planetary gears 303 via the carrier 305 is distributed to the sun gear 302 and the internally-toothed gear 301. At this time, in a case where no load is applied to the internally-toothed gear 301 and the sun gear 302, the internally-toothed gear 301 and the sun gear 302 rotate integrally with a revolution of the planetary gears 303 (a rotation of the carrier 305). In a case where no load is applied to the internally-toothed gear 301 but a load is applied to the sun gear 302, the sun gear 302 rotates at a speed reduction ratio according to the applied load. In a case where the sun gear 302 is fixed, the rotational power transmitted (input) to the planetary gears 303 is transmitted to the internally-toothed gear 301, and the internally-toothed gear 301 and the planetary gears 303 can continue their rotations. The planetary gear mechanism 300 included in the driving unit 200 can be embodied by a conventionally-known and commonly-used planetary gear mechanism. Therefore, a detailed description of the planetary gear mechanism 300 is omitted herein.

The driving force of the motor 108 is transmitted to the pinion gear 201. The pinion gear 201 is meshed with the first gear 202 so as to enable transmission of the driving force to the first gear 202.

The first clutch 203 is engaged with the first gear 202 and the first idler gear 205. The first clutch 203 allows and blocks transmission of the driving force between the first gear 202 and the first idler gear 205. More specifically, when the first clutch 203 is in an ON state, the driving force of the motor 108 is transmitted from the first gear 202 to the first idler gear 205 via the first clutch 203. On the other hand, when the first clutch 203 is in an OFF state, the driving force of the motor 108 is not transmitted to the first idler gear 205.

The second clutch 204 is engaged with the first gear 202 and the carrier gear 304 of the planetary gear mechanism 300. The second clutch 204 allows and blocks transmission of the driving force between the first gear 202 and the carrier gear 304. More specifically, when the second clutch 204 is in the ON state, the driving force of the motor 108 is transmitted from the first gear 202 to the carrier gear 304 via the second clutch 204. On the other hand, when the second clutch 204 is in the OFF state, the driving force of the motor 108 is not transmitted to the carrier gear 304.

The first idler gear 205 is meshed with the second idler gear 206 so as to enable transmission of the driving force of the motor 108 to the second idler gear 206. The second idler gear 206 is meshed with the sun gear 302 of the planetary gear mechanism 300 so as to enable transmission of the driving force of the motor 108 to the sun gear 302. The sun gear 302 of the planetary gear mechanism 300 is coupled to the conveyance screw 208 so as to enable transmission of the driving force of the motor 108 to the conveyance screw 208 via a conveyance screw gear 207.

### (First and Second Driving Force Transmission Units)

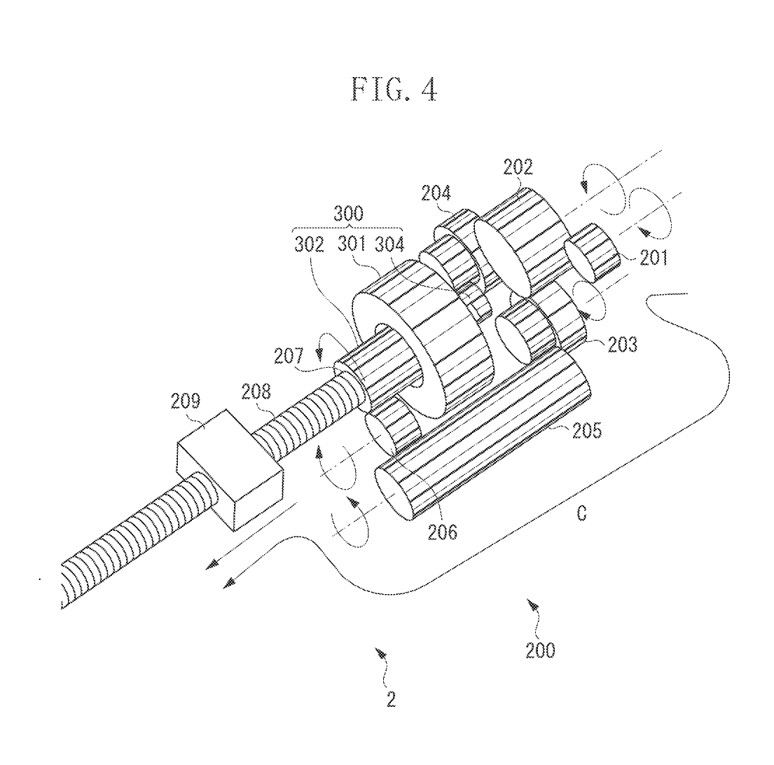

Next, operations of first and second driving force transmission units of the driving mechanism 2 disposed at the ophthalmologic apparatus 1 according to the present exemplary embodiment will be described with reference to FIGS. 4 to 6. Each of the states of the first clutch 203 and the second clutch 204 will be described.

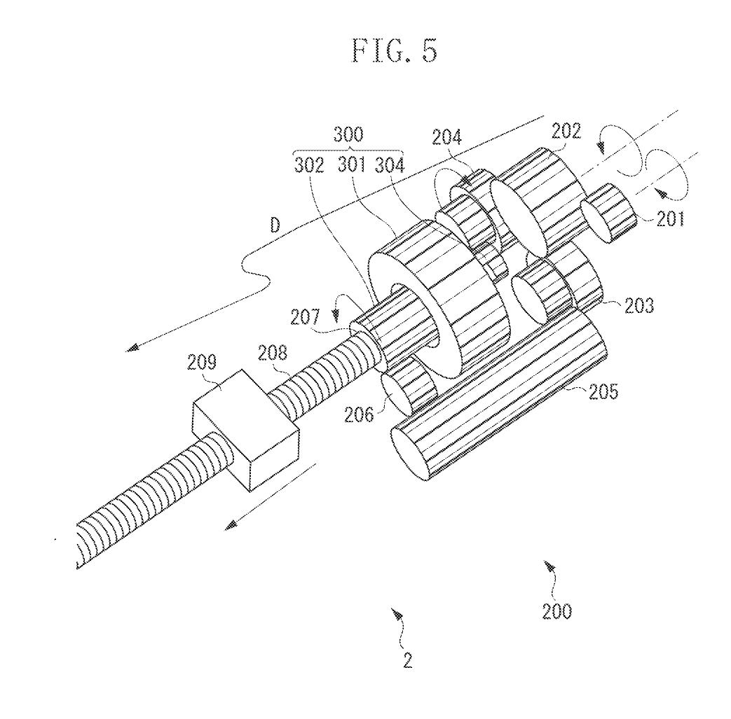

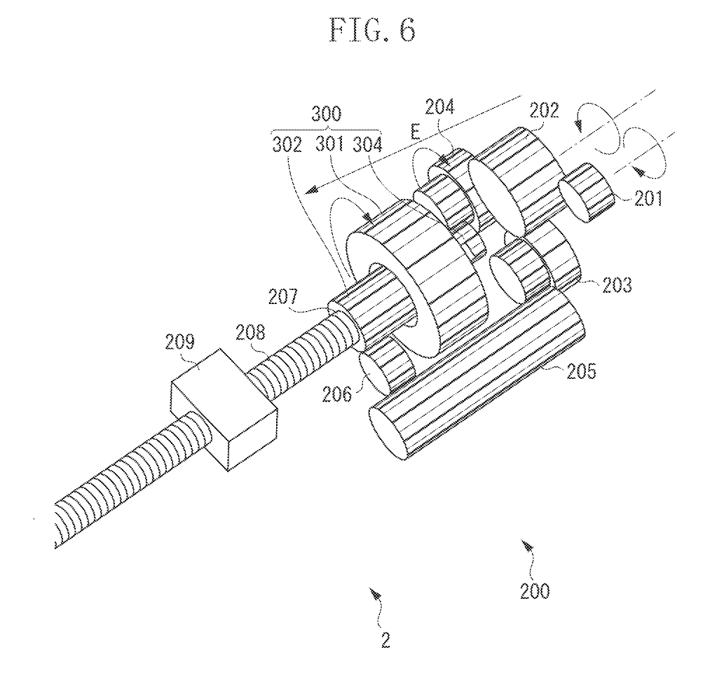

When the first clutch 203 is in the ON state and the second clutch 204 is in the OFF state, the driving mechanism 2 operates in the following manner. FIG. 4 is a perspective view schematically illustrating an operation of the driving mechanism 2 when the first clutch 203 is in the ON state and the second clutch 204 is in the OFF state. An arrow C in FIG. 4 indicates a path along which the driving force of the motor 108 is transmitted (a drive train). Further, other unlabeled arrows schematically indicate rotations of the respective gears. The gears with no arrow attached thereto indicate gears to which the driving force of the motor 108 is not transmitted (the same applies to FIGS. 5 and 6).

First, the driving force of the motor 108 is transmitted to the first clutch 203 and the second clutch 204 via the pinion gear 201 and the first gear 202. When the first clutch 203 is in the ON state, the driving force of the motor 108 is transmitted from the first clutch 203 to the conveyance screw 208 via the first idler gear 205, the second idler gear 206, the sun gear 302, and the conveyance screw gear 207. Then, the nut 209 moves in the Z-axis direction according to a rotation of the conveyance screw 208, and the Z movable table 107 moves in the Z-axis direction according to the movement of the nut 209. In this way, the driving unit 200 has the first driving force transmission unit that includes the first idler gear 205 and the second idler gear 206, via which the driving force of the motor 108 is transmitted to the conveyance screw 208. In the present specification, the first driving force transmission unit may be also referred to as a first drive train in some cases. At this time, since the second clutch 204 is in the OFF state, the driving force of the motor 108 is not transmitted to the carrier gear 304 and the planetary gears 303.

When the first clutch 203 is in the OFF state and the second clutch 204 is in the ON state, the driving mechanism 2 operates in the following manner. FIG. 5 is a perspective view schematically illustrating an operation of the driving mechanism 2 when the first clutch 203 is in the OFF state and the second clutch 204 is in the ON state. An arrow D in FIG. 5 indicates a path along which the driving force of the motor 108 is transmitted (a drive train).

First, the driving force of the motor 108 is transmitted to the first clutch 203 and the second clutch 204 via the pinion gear 201 and the first gear 202. When the second clutch 204 is in the ON state, the driving force of the motor 108 is transmitted from the second clutch 204 to the conveyance screw 208 via the carrier gear 304, the planetary gears 303, the sun gear 302, and the conveyance screw gear 207, as illustrated in FIG. 3B. Then, the nut 209 moves in the Z-axis direction according to a rotation of the conveyance screw 208, and the Z movable table 107 moves in the Z-axis direction according to the movement of the nut 209. In this way, the driving unit 200 has the second driving force transmission unit that includes the carrier gear 304 and the planetary gears 303, via which the driving force of the motor 108 is transmitted to the sun gear 302. In the present specification, the second driving force transmission unit may be also referred to as a second drive train in some cases. At this time, since the first clutch 203 is in the OFF state, the driving force of the motor 108 is not transmitted to the first idler gear 205 and the second idler gear 206.

When the sun gear 302 is fixed so as not to rotate with the first clutch 203 set in the OFF state and the second clutch 204 set in the ON state, the driving mechanism 2 operates in the following manner. FIG. 6 is a perspective view schematically illustrating an operation of the driving mechanism 2 when the sun gear 302 is fixed so as not to rotate with the first clutch 203 set in the OFF state and the second clutch set in the ON state. An arrow E illustrated in FIG. 6 schematically indicates a path along witch the driving force of the motor 108 is transmitted.

The sun gear 302 is coupled to the conveyance screw 208 via the conveyance screw gear 207. Therefore, when the Z movable table 107 stops moving due to application of an external force thereto, the sun gear 302 is fixed to prevent rotation. In this case, the planetary gear mechanism 300 transmits the rotational power to the internally-toothed gear 301 as a rotational power distribution mechanism. This operation will be described below more specifically.

As illustrated in FIG. 3C, since the sun gear 302 becomes a fixed gear, the driving force of the motor 108, which is transmitted to the carrier gear 304, is transmitted to the carrier 305 coupled to the carrier gear 304. Further, the rotational power input to the planetary gears 303 is transmitted to the internally-toothed gear 301. At this time, the internally-toothed gear 301 spins around idly without transmitting the driving force to another member since the internally-toothed gear 301 is not meshed with any driven gear. Therefore, when the Z movable table 107 stops moving due to application of an external force thereto (a movement of the Z movable table 107 is prevented), the driving force of the motor 108 is transmitted to the internally-toothed gear 301. As a result, the transmission of the driving force to the Z movable table 107 is cut off (the driving force transmitted to the Z movable table 107 reduces).

In this way, the driving unit 200 has the first driving force transmission unit and the second driving force transmission unit that transmit the driving force of the motor 108 to the conveyance screw 208. The first clutch 203 and the second clutch 204 switch the drive train between the first drive train and the second drive train (function as a switching unit). The second driving force transmission unit can be configured in any manner as long as it is configured to reduce the driving force transmitted to the movable table when an external force is applied to the movable table, and does not necessarily have to be configured not to transmit all the driving force. Further, the second driving force transmission unit may function to prevent the movable table from being driven in response to, for example, only a touch of an examiner. Therefore, the second driving force transmission unit is preferably used together with the first driving force transmission unit, as will be described below. Accordingly, the present invention can be realized only by the second driving force transmission unit, and the first driving force transmission unit may be omitted.

### (System Configuration of Opthalmologic Apparatus)

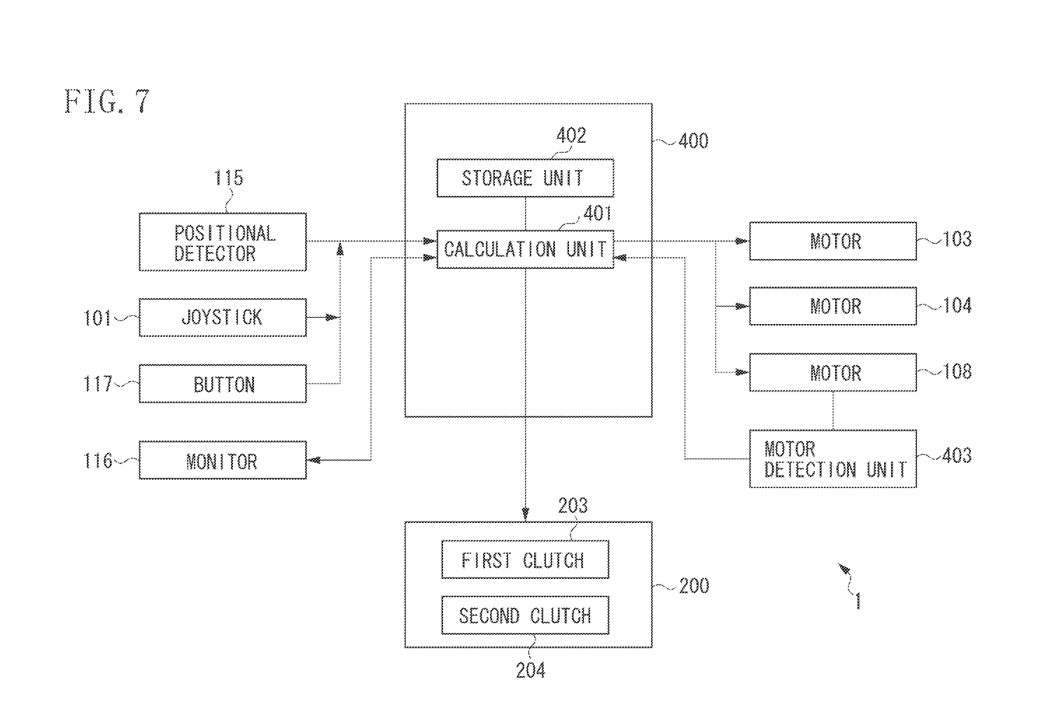

Next, a system configuration and an entire operation of the ophthalmologic apparatus 1 will be described with reference to FIG. 7. FIG. 7 is a block diagram schematically illustrating the system configuration of the ophthalmologic apparatus 1. The ophthalmologic apparatus 1 further includes a motor detection unit 403 as a movement direction detection unit, and a signal processing unit 400.

The motor detection unit 403 as the movement direction detection unit detects the number of rotations and a rotational direction of the motor 108. The motor detection unit 403 may be embodied by any of known types of revolution indicators. The motor detection unit 403 can detect a direction in which the Z movable table 107 moves, according to the rotational direction of the motor 108.

The signal processing unit 400 controls the entire ophthalmologic apparatus 1. The signal processing unit 400 includes a calculation unit 401 and a storage unit 402. The signal processing unit 400 is embodied by a computer including a calculation unit (a central processing unit (CPU)) and a storage medium. The calculation unit 401 detects an operation of the joystick 101 and an operation of the button 117, and controls the motors 103, 104, and 108 according to the detection result. Therefore, an examiner can make a positional adjustment of the measurement unit 110 such as an alignment adjustment of the measurement unit 110 by operating the joystick 101. The term “manual alignment” is used to indicate a positional adjustment of the measurement unit 110 that an examiner makes by operating the joystick 101. Further, the signal processing unit 400 can automatically make an alignment adjustment of the measurement unit 110 upon detection of pressing of the button 117 provided on the joystick 101. The term “automatic alignment” is used to indicate a positional adjustment of the measurement unit 110 that is made automatically.

The calculation unit 401 receives a detection result by the motor detection unit 403 about the number of rotations and a rotational direction of the motor 108, and a detection result by the position detector 115 about a position of the Z movable table 107. Then, the calculation unit 401 determines whether an external force is applied to the Z movable table 107 to prevent a movement of the Z movable table 107 (or stop a movement of the Z movable table 107) based on the detection result by the motor detection unit 403 and the detection result by the position detector 115 (as will be described below).

Further, the calculation unit 401 determines whether a distance between the measurement unit 110 and a subject (a subject's eye) in the Z-axis direction is equal to or smaller than a predetermined threshold value (whether the measurement unit 110 is located close to or away from the subject (the subject's eye)) based on the detection result by the position detector 115. This predetermined threshold value is stored in the storage unit 402 in advance. The calculation unit 401 is configured to use an initial value of the predetermined threshold value stored in the storage unit 402 in advance. Instead of it, the calculation unit 401 may be configured to use a predetermined threshold value arbitrarily set or selected by the examiner.

In a case where the distance between the Z movable table 107 and the subject (the subject's eye) is larger than the predetermined threshold value, the calculation unit 401 transmits an ON signal to the first clutch 203, and transmits an OFF signal to the second clutch 204. In response to these signals, the first clutch 203 is set into the ON state, in which the first clutch 203 allows transmission of the driving force of the motor 108, and the second clutch 204 is set into the OFF state, in which the second clutch 204 blocks transmission of the driving force of the motor 108. As a result, the driving force of the motor 108 is transmitted to the conveyance screw 208 via the first idler gear 205 and the second idler gear 206 (i.e., the first drive train), thereby causing the Z movable table 107 to move (refer to FIG. 4).

On the other hand, in a case where the distance between the Z movable table 107 and the subject (the subject's eye) is equal to or smaller than the predetermined threshold value, the calculation unit 401 transmits an OFF signal to the first clutch 203, and transmits an ON signal to the second clutch 204. In response to these signals, the first clutch 203 is set into the OFF state, and the second clutch 204 is set into the ON state. As a result, the driving force of the motor 108 is transmitted to the conveyance screw 208 via the carrier gear 304 and the planetary gears 303 (the second drive train), thereby causing the Z movable table 107 to move (refer to FIG. 5).

In this way, in a case where the measurement unit 110 is located away from the chin support table 112 and the fixed table 100 in the Z-axis direction, the first clutch 203 and the second clutch 204 switch the driving unit 200 to such a state that the driving unit 200 transmits the driving force via the first drive train. In this state, the driving force of the motor 108 is transmitted to the conveyance screw 208 without an intervention of the carrier gear 304 and the planetary gears 300 of the planetary gear mechanism 300. On the other hand, in a case where the measurement unit 110 is located close to the chin support table 112 and the fixed table 100, the first clutch 203 and the second clutch 204 switch the driving unit 200 to such a state that the driving unit 200 transmits the driving force via the second drive train. According to this configuration, it is detected whether a limb or the like of the subject is caught between the measurement unit 100 and the chin support table 112 and the fixed table 100, only when the measurement unit 110 is located close to the chin support table 112 and the fixed table 100 (as will be described below).

Whether the measurement unit 110 is located away from the subject (the subject's eye) is determined based on whether the distance between the measurement unit 110 and the subject (the subject's eye) in the Z-axis direction is equal to or smaller than the predetermined threshold value. This predetermined threshold value is arbitrarily set according to the specific configuration of the measurement unit 110, the Z movable table 107, the fixed table 100, and the like.

### (Operation Flow of Driving Unit at the Time of Movement in Z-Axis Direction)

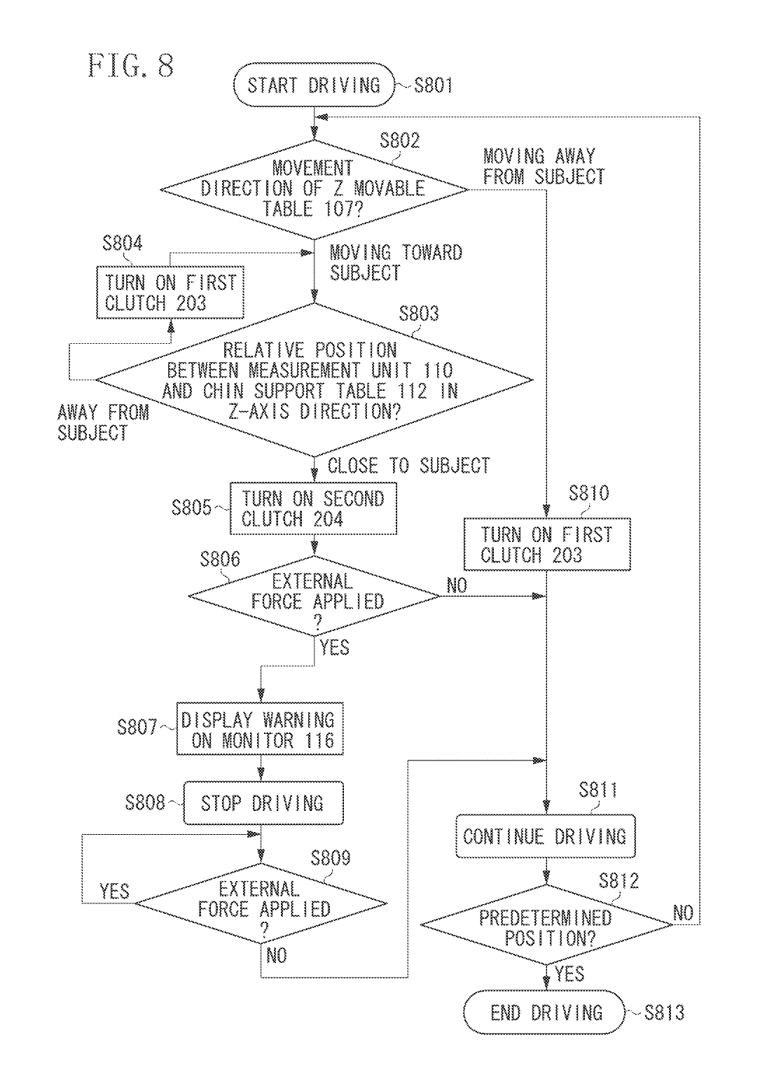

Next, a flow of an operation that the driving unit 200 of the ophthalmologic apparatus 1 performs to move the Z movable table 107 in the Z-axis direction will be described with reference to a flowchart of FIG. 8, based on an actual flow of an eye examination. FIG. 8 is a flowchart illustrating an operation and processing of the ophthalmologic apparatus 1 when the Z movable table 107 moves in the Z-axis direction.

In step S801, the calculation unit 401 of the signal processing unit 400 starts driving the Z movable table 107. Upon detection of an operation of the joystick 101 for moving the Z movable table 107 in the Z-axis direction, the calculation unit 401 drives the motor 108. As a result, the measurement unit 110 starts moving in the Z-axis direction together with the Z movable table 107. Then, the processing proceeds to step S802.

In step S802, the calculation unit 401 determines whether the movement direction of the Z movable table 107 detected by the motor detection unit 403 is a direction moving toward a subject (a subject's eye) or moving away from the subject (the subject's eye). Then, if the movement direction of the Z movable table 107 is the direction moving toward the subject (the subject's eye) (MOVING TOWARD SUBJECT in step S802), the processing proceeds to step S803. If the movement direction of the Z movable table 107 is the direction moving away from the subject (the subject's eye) (MOVING AWAY FROM SUBJECT in step S802), the processing proceeds to step S810.

Steps S803 to S809 are an operation and processing in a case where the Z movable table 107 moves in the direction toward the subject (the subject's eye), for example, an operation and processing performed before a start of an eye examination. An examiner adjusts a position of the chin support table 112 in the Y-axis direction in such a manner that an anterior eye portion of the subject's eye is displayed on the monitor 116 while the subject rests his/her head on the chin support table 112, before a start of an eye examination. Next, the examiner makes a positional adjustment so that the subject's eye is positioned at a correct position for measurement. This positional adjustment includes an operation and processing for moving the Z movable table 107 in the Z-axis direction to approach the subject (the subject's eye) based on an operation of the examiner. This positional adjustment may be the automatic alignment or may be the manual alignment. The present exemplary embodiment is described here based on an example of a positional adjustment by the manual alignment.

In the exemplary embodiment of the present invention, a non-contact tonometer is employed as the ophthalmologic apparatus 1. In the non-contact tonometer, a distance between the measurement unit 110 and a subject's eye at the time of an eye examination is as small as approximately 10 mm. Therefore, before the eye examination, the examiner makes the measurement unit 110 waiting at a position away from the subject's eye. At the time of the eye examination, after the subject puts his/her head on the chin support table 112, the examiner starts moving the measurement unit 110 toward the subject's eye in the Z-axis direction. Further, in the exemplary embodiment of the present invention, the ophthalmologic apparatus 1 is a non-contact tonometer, but the present invention can also be applied to another ophthalmologic apparatus as long as the ophthalmologic apparatus has a possibility that a limb or the like of a subject may be caught between a movable unit and a fixed unit.

In step S803, the calculation unit 401 determines whether a distance between the measurement unit 110 and the subject (the subject's eye) is equal to or smaller than the predetermined threshold value based on a detection result of the position detector 115 and the predetermined threshold value stored in the storage unit 402 in advance. Then, if this distance is not equal to or smaller than the predetermined threshold value (if the measurement unit 110 is located away from the subject (the subject's eye)) (AWAY FORM SUBJECT instep S803), the processing proceeds to step S804. On the other hand, if the distance is equal to or smaller than the predetermined threshold value (if the measurement unit 110 is located close to the subject (the subject's eye)) (CLOSE TO SUBJECT in step S803), the processing proceeds to step S805.

In step S804, the calculation unit 401 transmits an ON signal to the first clutch 203, and transmits an OFF signal to the second clutch 204. Therefore, the first clutch 203 and the second clutch 204 switch the driving unit 200 to the state in which the driving unit 200 transmits the driving force of the motor 108 via the first drive train. In a case where the driving unit 200 is already switched to this state, the first clutch 203 and the second clutch 204 maintain this state. Then, steps S803 and S804 are repeated until the calculation unit 401 determines that the distance between the measurement unit 110 and the subject (the subject's eye) is equal to or smaller than the predetermined threshold value. If the calculation unit 401 determines that the distance between the measurement unit 110 and the subject (the subject's eye) is equal to or smaller than the predetermined threshold value, the processing proceeds to step S805.

In step S805, the calculation unit 401 transmits an OFF signal to the first clutch 203, and transmits an ON signal to the second clutch 204. Therefore, the first clutch 203 and the second clutch 204 switch the driving unit 200 to the state in which the driving unit 200 transmits the driving force of the motor 108 via the second drive train.

Before the start of the eye examination, the measurement unit 110 is located at a position away from the subject (the subject's eye) (away from the subject by a distance at least larger than the predetermined threshold value). In this state, the first clutch 203 and the second clutch 204 switch the driving unit 200 to the state in which the driving unit 200 transmits the driving force of the motor 108 via the first drive train (or maintain this state). Then, while keeping this state, the measurement unit 110 and the Z movable table 107 continue moving in the direction toward the subject (the subject's eye) along the Z-axis direction, whereby the distance between measurement unit 110 and the subject (the subject's eye) becomes equal to or smaller than the predetermined threshold value. When this distance becomes equal to or smaller than the predetermined threshold value, the first clutch 203 and the second clutch 204 switch the driving unit 200 to the state in which the driving unit 200 transmits the driving force of the motor 108 via the second drive train. Then, the processing proceeds to step S806.

In step S806, the calculation unit 401 determines whether an external force is applied to the measurement unit 110 or the Z movable table 107 to prevent their movements in the Z-axis direction(or stop their movements).

The calculation unit 401 determines whether an external force is applied in the following manner. Before the start of the eye examination, the measurement unit 110 is located away from the subject (the subject's eye). At this time, the examiner may operate the ophthalmologic apparatus 1 to cause the measurement unit 110 to move toward the subject (the subject's eye) without noticing that the subject puts his/her hand or the like between the measurement unit 110, and the chin support table 112 and the fixed table 100. In this case, due to a movement of the measurement unit 110 toward the subject (the subject's eye), the subject's hand or the like may be caught between the measurement unit 110, and the chin support table 112 or the fixed table 100. In a case where the subject's hand or the like is caught between the measurement unit 110, and the chin support table 112 or the fixed table 100, the measurement unit 110 and the Z movable table 107 cannot move toward the subject any more. Therefore, the nut 209 cannot move, and the conveyance screw 208 and the sun gear 302 stops rotating. As a result, the driving force of the motor 108 is transmitted to the internally-toothed gear 301 of the planetary gear mechanism 300 and is not transmitted to the sun gear 302 (refer to FIG. 6).

When the driving force of the motor 108 is normally transmitted to the sun gear 302, the number of rotations of the motor 108 and the displacement amount of the Z movable table 107 are in a proportional relationship. On the other hand, when the internally-toothed gear 301 rotates by the driving force of the motor 108, the number of rotations of the motor 108 and the displacement amount of the Z movable table 107 are not in the above-described proportional relationship. Therefore, the calculation unit 401 detects whether the number of rotations of the motor 108 and the displacement amount of the Z movable table 107 are in the proportional relationship based on a detection result of the motor detection unit 403 and a detection result of the position detector 115. The calculation unit 401 determines that an external force is applied to the measurement unit 110 and the Z movable table 107 to prevent their movements (or stop their movements) in a case where the number of rotations of the motor 108 and the displacement amount of the Z movable table 107 are not in the proportional relationship.

The patent literature described above as a conventional art discusses the unit for preventing an abnormal approach with use of a unit for detecting positions of the measurement unit and the subject's eye, but it is difficult to detect occurrence of such a situation that the subject's hand or the like is caught by this technique.

If the calculation unit 401 determines that an external force is applied to the measurement unit 110 and the Z movable table 107 (YES instep S806), the processing proceeds to step S807. On the other hand, if the calculation unit 401 determines that an external force is not applied to the measurement unit 110 and the Z movable table 107 (NO in step S806), the processing proceeds to step S811.

In step S807, the calculation unit 401 controls the monitor 116, and the monitor 116 displays a notification indicating that an external force is applied to the measurement unit 110 and the Z movable table 107. Then, the processing proceeds to step S808. In step S808, the calculation unit 401 stops the rotation of the motor 108. The orders of steps S807 and S808 maybe reversed, or steps S807 and S808 maybe performed simultaneously. Then, the processing proceeds to step S809.

In step S809, the calculation unit 401 determines whether an external force is applied to the measurement unit 110 and the Z movable table 107. The determination method is the same as step S806. If the calculation unit 401 determines that an external force is applied (YES in step S809), as illustrated in FIG. 3C, the planetary gear mechanism 300 works as a rotational power distribution mechanism, so the rotational power is transmitted to the internally-toothed gear 301 to cause the internally-toothed gear 301 to spin around idly. Therefore, the measurement unit 110 stops or slows down its movement. Then, step S809 is repeated until the calculation unit 401 determines that an external force is not applied. If the calculation unit 401 determines that an external force is not applied in step S809 (NO in step S809), the processing proceeds to step S811.

In step S811, the calculation unit 401 continues (restarts) driving the motor 108. Then, the processing proceeds to step S812.

In step S812, the calculation unit 401 determines whether the measurement unit 110 has reached a predetermined position. If the calculation unit 401 determines that the measurement unit 110 has reached the predetermined position (YES in step S812), the processing proceeds to step S813, in which the calculation unit 401 ends driving the motor 108. If the calculation unit 401 determines that the measurement unit 110 has not reached the predetermined position (NO in step S812), the processing proceeds to step S802 again, and step S802 and the steps thereafter are repeated.

The predetermined distance described here is a distance between the measurement unit 110 and the subject's eye when the measurement unit 110 examines the subject's eye. This predetermined distance is also stored in the storage unit 402 in advance.

Referring back to step S802, the flowchart will be further described. In step S802, if the calculation unit 401 determines that the Z movable table 107 is moving away from the subject (the subject's eye) (MOVING AWAY FROM SUBJECT in step S802), the processing proceeds to step S810.

In step S810, the calculation unit 401 transmits an ON signal to the first clutch 203, and transmits an OFF signal to the second clutch 204. Therefore, the first clutch 203 and the second clutch 204 switch the driving unit 200 to the state in which the driving unit 200 transmits the driving force of the motor 108 via the first drive train. Then, the processing proceeds to step S811.

In step S811, the calculation unit 401 continues driving the motor 108. Then, the processing proceeds to step S812. In step S812, the calculation unit 401 determines whether the measurement unit 110 has reached the predetermined position. If the calculation unit 401 determines that the measurement unit 110 has reached the predetermined position (YES in step S812), the processing proceeds to step S813, in which the calculation unit 401 ends driving the motor 108. If the calculation unit 401 determines that the measurement unit 110 has not reached the predetermined position (NO in step S812), the processing proceeds to step S802 again, and step S802 and the steps thereafter are repeated. The predetermined distance described here is a distance when the measurement unit 110 is located away from the subject (the subject's eye) (a distance at least larger than the above-described predetermined threshold value). This predetermined distance is also stored in the storage unit 402 in advance.

After completion of the eye examination, the examiner causes the measurement unit 110 to move to a position away from the subject (the subject's eye) in the Z-axis direction for an eye examination of a next subject. In this case, it is unlikely that a limb of the subject or the examiner would be caught between the chin support table 112 and the measurement unit 110. Therefore, if the calculation unit 401 determines that the Z movable table 107 is moving away from the subject (the subject's eye), the first clutch 203 and the second clutch 204 switch the driving unit 200 to the state in which the driving unit 200 transmits the driving force of the motor 108 via the first drive train. In this case, even when an external force is applied to the measurement unit 110 and the Z movable table 107, the calculation unit 401 does not stop driving the motor 108.

For example, the examiner may cause the Z movable table 107 to move while performing an operation such as pressing a touch panel on the monitor 116 after completion of the eye examination. In this case, if the driving unit 200 is switched to the state in which the driving unit 200 transmits the driving force via the second drive train, the calculation unit 401 may mistake the force that the examiner applies onto the motor 116 or the like (for example, a force touching the touch panel), for an external force that prevents a movement of the measurement unit 110. Therefore, in a case where the calculation unit 401 determines that the Z movable table 107 is moving away from the subject (the subject's eye), the first clutch 203 and the second clutch 204 switch the driving unit 200 to the state in which the driving unit 200 transmits the driving force of the motor 108 via the first drive train. Thus, it is possible to prevent the above-described false detection.

The present exemplary embodiment has been described above in detail. However, the present exemplary embodiment merely indicates one specific example of how the present invention is embodied. The technical range of the present invention is not limited to the present exemplary embodiment. The present invention can be variously modified within the scope of the present invention, and such modifications are also included in the technical range of the present invention.

For example, the present exemplary embodiment has been described based on a configuration in which the ophthalmologic apparatus is a non-contact tonometer. However, the type of the ophthalmologic apparatus is not limited thereto. The present invention can be applied to any ophthalmologic apparatus capable of measuring a predetermined item of a subject's eye and having a unit movable toward and away from the subject's eye, and such an embodiment is also included in the technical range of the present invention.

Further, in the present exemplary embodiment, the driving is switched at the time of application of an external force by using the planetary gear mechanism. Alternatively, the driving can be switched by using a direct-current (DC) motor as a driving source. In that case, a current flowing through the DC motor is monitored, and application of an external force can be detected based on this monitoring. It is well known that a DC motor has such a characteristic that a change in a load causes a change in a flowing current. The DC motor can be used as an external force detection sensor using this characteristic.

For example, when the measurement unit 110 starts measurement at a position away from a subject, the position of the measurement unit 110 is adjusted relative to the position of the subject's eye. After a rise time immediately after a start of a movement of the Z movable table 107 has elapsed, the Z movable table 107 moves at a substantially constant speed, and therefore a substantially constant current is supplied to the DC motor. After that, in a case where an external force is applied while the Z movable table 107 is supposed to move at the substantially constant speed according to a signal from a main body control unit (not illustrated), the current supplied to the DC motor increases. Therefore, it is determined that an external force is applied if the increased current value is larger than a threshold value stored in the main body in advance. Then, current supply from the main body control unit to the DC motor and the driving are stopped. Further, a warning indicating a stop due to application of an external force is displayed on the monitor 116.

This external force detection control cannot be employed when a load largely changes, for example, immediately after driving of the DC motor starts or immediately before driving of the DC motor stops, or when the number of rotations of the DC motor largely changes during a positional adjustment. Therefore, a range where an external force is detected using the DC motor is a range from the time when the number of rotations of the DC motor becomes substantially constant after the DC motor starts to rotate, until the time when the distance from the subject's eye to the measurement unit 110 falls below a threshold value stored in the main body in advance.

Further, in the manual alignment, it is determined that the Z movable table 107 is moving at a substantially constant speed when the Z movable table 107 moves at the substantially constant speed for a time stored in the main body in advance, or for a longer time. Then, detection of an external force by the DC motor is started.

Further, the present exemplary embodiment has been described based on a configuration in which the ophthalmologic apparatus 1 includes the position detector 115 as the position detection unit, and the position detector 115 detects the distance between the measurement unit 110 as the ophthalmologic examination unit and the subject's eye. However, the present invention does not necessarily have to be configured in this manner. For example, another embodiment of the present invention may be configured such that the position detector 115 detects only a position of the measurement unit 110, and the calculation unit 401 detects (calculates) a distance between the measurement unit 110 and the subject's eye based on the detection result of the position detector 115. In other words, another exemplary embodiment may be configured such that the position detector 115 and the calculation unit 401 cooperate to function as the position detection unit. In this way, the present invention may be configured in such a manner that each unit is realized by a single hardware device, or each unit is realized by a plurality of hardware devices.

The present exemplary embodiment is a useful technique for an ophthalmologic apparatus capable of measuring a predetermined item of a subject's eye, and including a unit movable toward and away from the subject's eye. According to the present invention, it is possible to detect that a limb or the like of a subject or an examiner is caught between a movable unit and a fixed unit.

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment (s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all modifications, equivalent structures, and functions.

This application claims priority from Japanese Patent Application No. 2012-105364 filed May 2, 2012, and No. 2013-066883 filed Mar. 27, 2013, which are hereby incorporated by reference herein in their entirety.

What is claimed is:

1. An ophthalmologic apparatus comprising:
an examination unit configured to examine a subject's eye;
a movable unit configured to move the examination unit;
a driving force generation unit configured to generate a driving force to drive the movable unit; and
a driving force transmission unit configured to reduce the driving force transmitted to the movable unit in a case where an external force is applied to the movable unit.

2. The ophthalmologic apparatus according to claim 1, wherein the driving force transmission unit transmits the driving force to the movable unit in a case where the external force is not applied to the movable unit, and the driving force transmission unit does not transmit the driving force to the movable unit in a case where the external force is applied to the movable unit.

3. The ophthalmologic apparatus according to claim 1, further comprising:
a first driving force transmission unit configured to transmit the driving force to the movable unit; and
a switching unit configured to switch between the first driving force transmission unit and a second driving force transmission unit which also transmits the driving force to the movable unit, according to a state of the movable unit relative to the subject's eye.

4. The ophthalmologic apparatus according to claim 3, wherein the driving force is transmitted to the movable unit by the second driving force transmission unit to which the first driving force transmission unit is switched by the switching unit, in a case where the movable unit is moving toward the subject' s eye and a distance between the subject' s eye and the movable unit is equal to or smaller than a threshold value.

5. The ophthalmologic apparatus according to claim 3, wherein the switching unit is configured to switch between the first driving force transmission unit and the second driving force transmission unit by switching between a first clutch and a second clutch, and the second driving force transmission unit includes a carrier gear; and
Wherein the driving force is transmitted to the carrier gear in a case where the second clutch is in an ON state, and the driving force is not transmitted to the carrier gear in a case where the second clutch is in an OFF state.

6. The ophthalmologic apparatus according to claim 5, wherein the second driving force transmission unit includes a planetary gear mechanism that has a planetary gear to which the driving force is transmitted from the driving force generation unit, and a sun gear meshing with the planetary gear and configured to transmit the driving force to the movable unit is fixed to prevent its rotation in a case where the second clutch is in an ON state.

7. The ophthalmologic apparatus according to claim 3, wherein the switching unit switches the driving force transmission method from the second driving force transmission unit to the first driving force transmission unit so that the first driving force transmission unit transmits the driving force to the movable unit, in a case where the movable unit is moving away from the subject's eye.

8. The ophthalmologic apparatus according to claim 3, further comprising:
a movement direction detection unit configured to detect a movement direction of the movable unit; and
a position detection unit configured to detect a distance between the movable unit and the subject's eye,
wherein the switching unit switches the driving force transmission method to the second driving force transmission unit in a case where the movement direction of the movable unit detected by the movement direction detection unit, is a direction toward the subject's eye, and the distance between the movable unit and the subject's eye detected by the position detection unit, is equal to or smaller than a threshold value.

9. The ophthalmologic apparatus according to claim 8, wherein the switching unit switches the driving force transmission method to the first driving force transmission unit in a case where the movement direction of the movable unit detected by the movement direction detection unit, is a direction toward the subject's eye, and the distance between the movable unit and the subject's eye detected by the position detection unit, is larger than the threshold value.

10. The ophthalmologic apparatus according to claim 8, wherein the switching unit switches the driving force transmission method to the first driving force transmission unit in a case where the movement direction of the movable unit detected by the movement direction detection unit, is a direction away from the subject eye.

11. The ophthalmologic apparatus according to claim 1, further comprising:
a display control unit configured to cause a display unit to display a warning that the reduction of the driving force has been caused by the the external force, in a case where the external force is applied to the movable unit

12. A method for controlling an ophthalmologic apparatus, comprising:
generating a driving force to drive a movable unit configured to move an examination unit configured to examine a subject's eye; and
reducing the driving force transmitted to the movable unit in a case where an external force is applied to the movable unit.

13. The method for controlling an ophthalmologic apparatus according to claim 12, further comprising:
transmitting the driving force to the movable unit in a case where the external force is not applied to the movable unit; and
not transmitting the driving force to the movable unit during the reduction of the driving force in a case where the external force is applied to the movable unit.

14. A storage medium storing a program for causing a computer to execute the method for controlling the ophthalmologic apparatus according to claim 12.
